SEQUENCE LISTING (10 sequences)

SEQ ID NO: 1 - DNA, 993 bp, Escherichia coli
  CDS (1)..(990): rpos gene
atgagtcagaatacgctgaaagttcatgatttaaatgaagatgcggaatttgatgagaacggagttgaggtttttgacgaaaaggccttagtagaacaggaacccagtgataacgatttggccgaagaggaactgttatcgcagggagccacacagcgtgtgttggacgcgactcagctttaccttggtgagattggttattcaccactgttaacggccgaagaagaagtttattttgcgcgtcgcgcactgcgtggagatgtcgcctctcgccgccggatgatcgagagtaacttgcgtctggtggtaaaaattgcccgccgttatggcaatcgtggtctggcgttgctggaccttatcgaagagggcaacctggggctgatccgcgcggtagagaagtttgacccggaacgtggtttccgcttctcaacatacgcaacctggtggattcgccagacgattgaacgggcgattatgaaccaaacccgtactattcgtttgccgattcacatcgtaaaggagctgaacgtttacctgcgaaccgcacgtgagttgtcccataagctggaccatgaaccaagtgcggaagagatcgcagagcaactggataagccagttgatgacgtcagccgtatgcttcgtcttaacgagcgcattacctcggtagacaccccgctgggtggtgattccgaaaaagcgttgctggacatcctggccgatgaaaaagagaacggtccggaagataccacgcaagatgacgatatgaagcagagcatcgtcaaatggctgttcgagctgaacgccaaacagcgtgaagtgctggcacgtcgattcggtttgctggggtacgaagcggcaacactggaagatgtaggtcgtgaaattggcctcacccgtgaacgtgttcgccagattcaggttgaaggcctgcgccgtttgcgcgaaatcctgcaaacgcaggggctgaatatcgaagcgctgttccgcgagtaa

SEQ ID NO: 2 - protein, 330 aa, Escherichia coli
MSQNTLKVHDLNEDAEFDENGVEVFDEKALVEQEPSDNDLAEEELLSQGATQRVLDATQLYLGEIGYSPLLTAEEEVYFARRALRGDVASRRRMIESNLRLVVKIARRYGNRGLALLDLIEEGNLGLIRAVEKFDPERGFRFSTYATWWIRQTIERAIMNQTRTIRLPIHIVKELNVYLRTARELSHKLDHEPSAEEIAEQLDKPVDDVSRMLRLNERITSVDTPLGGDSEKALLDILADEKENGPEDTTQDDDMKQSIVKWLFELNAKQREVLARRFGLLGYEAATLEDVGREIGLTRERVRQIQVEGLRRLREILQTQGLNIEALFRE

SEQ ID NO: 3 - DNA, 993 bp, Escherichia coli
  Allele (1)..(990): rpoS allele
  misc_feature (97)..(99): amber codon
atgagtcagaatacgctgaaagttcatgatttaaatgaagatgcggaatttgatgagaacggagttgaggtttttgacgaaaaggccttagtagaataggaacccagtgataacgatttggccgaagaggaactgttatcgcagggagccacacagcgtgtgttggacgcgactcagctttaccttggtgagattggttattcaccactgttaacggccgaagaagaagtttattttgcgcgtcgcgcactgcgtggagatgtcgcctctcgccgccggatgatcgagagtaacttgcgtctggtggtaaaaattgcccgccgttatggcaatcgtggtctggcgttgctggaccttatcgaagagggcaacctggggctgatccgcgcggtagagaagtttgacccggaacgtggtttccgcttctcaacatacgcaacctggtggattcgccagacgattgaacgggcgattatgaaccaaacccgtactattcgtttgccgattcacatcgtaaaggagctgaacgtttacctgcgaaccgcacgtgagttgtcccataagctggaccatgaaccaagtgcggaagagatcgcagagcaactggataagccagttgatgacgtcagccgtatgcttcgtcttaacgagcgcattacctcggtagacaccccgctgggtggtgattccgaaaaagcgttgctggacatcctggccgatgaaaaagagaacggtccggaagataccacgcaagatgacgatatgaagcagagcatcgtcaaatggctgttcgagctgaacgccaaacagcgtgaagtgctggcacgtcgattcggtttgctggggtacgaagcggcaacactggaagatgtaggtcgtgaaattggcctcacccgtgaacgtgttcgccagattcaggttgaaggcctgcgccgtttgcgcgaaatcctgcaaacgcaggggctgaatatcgaagcgctgttccgcgagtaa

SEQ ID NO: 4 - DNA, 75 bp, Escherichia coli
  tRNA (1)..(75): supE allele
tggggtatcgccaagcggtaaggcaccggattctaattccggcattccgaggttcgaatcctcgtaccccagcca

SEQ ID NO: 5 - DNA, 1545 bp, Escherichia coli
  CDS (1)..(1542): ilvA-Gen
atggctgactcgcaacccctgtccggtgctccggaaggtgccgaatatttaagagcagtgctgcgcgcgccggtttacgaggcggcgcaggttacgccgctacaaaaaatggaaaaactgtcgtcgcgtcttgataacgtcattctggtgaagcgcgaagatcgccagccagtgcacagctttaagctgcgcggcgcatacgccatgatggcgggcctgacggaagaacagaaagcgcacggcgtgatcactgcttctgcgggtaaccacgcgcagggcgtcgcgttttcttctgcgcggttaggcgtgaaggccctgatcgttatgccaaccgccaccgccgacatcaaagtcgacgcggtgcgcggcttcggcggcgaagtgctgctccacggcgcgaactttgatgaagcgaaagccaaagcgatcgaactgtcacagcagcaggggttcacctgggtgccgccgttcgaccatccgatggtgattgccgggcaaggcacgctggcgctggaactgctccagcaggacgcccatctcgaccgcgtatttgtgccagtcggcggcggcggtctggctgctggcgtggcggtgctgatcaaacaactgatgccgcaaatcaaagtgatcgccgtagaagcggaagactccgcctgcctgaaagcagcgctggatgcgggtcatccggttgatctgccgcgcgtagggctatttgctgaaggcgtagcggtaaaacgcatcggtgacgaaaccttccgtttatgccaggagtatctcgacgacatcatcaccgtcgatagcgatgcgatctgtgcggcgatgaaggatttattcgaagatgtgcgcgcggtggcggaaccctctggcgcgctggcgctggcgggaatgaaaaaatatatcgccctgcacaacattcgcggcgaacggctggcgcatattctttccggtgccaacgtgaacttccacggcctgcgctacgtctcagaacgctgcgaactgggcgaacagcgtgaagcgttgttggcggtgaccattccggaagaaaaaggcagcttcctcaaattctgccaactgcttggcgggcgttcggtcaccgagttcaactaccgttttgccgatgccaaaaacgcctgcatctttgtcggtgtgcgcctgagccgcggcctcgaagagcgcaaagaaattttgcagatgctcaacgacggcggctacagcgtggttgatctctccgacgacgaaatggcgaagctacacgtgcgctatatggtcggcggacgtccatcgcatccgttgcaggaacgcctctacagcttcgaattcccggaatcaccgggcgcgctgctgcgcttcctcaacacgctgggtacgtactggaacatttctttgttccactatcgcagccatggcaccgactacgggcgcgtactggcggcgttcgaacttggcgaccatgaaccggatttcgaaacccggctgaatgagctgggctacgattgccacgacgaaaccaataacccggcgttcaggttctttttggcgggttag

SEQ ID NO: 6 - protein, 514 aa, Escherichia coli [sequence omitted]

SEQ ID NO: 7 - DNA, 1545 bp, Escherichia coli
  CDS (1)..(1542): ilvA-Allel
  mutation (856)..(856)
atggctgactcgcaacccctgtccggtgctccggaaggtgccgaatatttaagagcagtgctgcgcgcgccggtttacgaggcggcgcaggttacgccgctacaaaaaatggaaaaactgtcgtcgcgtcttgataacgtcattctggtgaagcgcgaagatcgccagccagtgcacagctttaagctgcgcggcgcatacgccatgatggcgggcctgacggaagaacagaaagcgcacggcgtgatcactgcttctgcgggtaaccacgcgcagggcgtcgcgttttcttctgcgcggttaggcgtgaaggccctgatcgttatgccaaccgccaccgccgacatcaaagtcgacgcggtgcgcggcttcggcggcgaagtgctgctccacggcgcgaactttgatgaagcgaaagccaaagcgatcgaactgtcacagcagcaggggttcacctgggtgccgccgttcgaccatccgatggtgattgccgggcaaggcacgctggcgctggaactgctccagcaggacgcccatctcgaccgcgtatttgtgccagtcggcggcggcggtctggctgctggcgtggcggtgctgatcaaacaactgatgccgcaaatcaaagtgatcgccgtagaagcggaagactccgcctgcctgaaagcagcgctggatgcgggtcatccggttgatctgccgcgcgtagggctatttgctgaaggcgtagcggtaaaacgcatcggtgacgaaaccttccgtttatgccaggagtatctcgacgacatcatcaccgtcgatagcgatgcgatctgtgcggcgatgaaggatttattcgaagatgtgcgcgcggtggcgaaaccctctggcgcgctggcgctggcgggaatgaaaaaatatatcgccctgcacaacattcgcggcgaacggctggcgcatattctttccggtgccaacgtgaacttccacggcctgcgctacgtctcagaacgctgcgaactgggcgaacagcgtgaagcgttgttggcggtgaccattccggaagaaaaaggcagcttcctcaaattctgccaactgcttggcgggcgttcggtcaccgagttcaactaccgttttgccgatgccaaaaacgcctgcatctttgtcggtgtgcgcctgagccgcggcctcgaagagcgcaaagaaattttgcagatgctcaacgacggcggctacagcgtggttgatctctccgacgacgaaatggcgaagctacacgtgcgctatatggtcggcggacgtccatcgcatccgttgcaggaacgcctctacagcttcgaattcccggaatcaccgggcgcgctgctgcgcttcctcaacacgctgggtacgtactggaacatttctttgttccactatcgcagccatggcaccgactacgggcgcgtactggcggcgttcgaacttggcgaccatgaaccggatttcgaaacccggctgaatgagctgggctacgattgccacgacgaaaccaataacccggcgttcaggttctttttggcgggttag

SEQ ID NO: 8 - protein, 514 aa, Escherichia coli
MADSQPLSGAPEGAEYLRAVLRAPVYEAAQVTPLQKMEKLSSRLDNVILVKREDRQPVHSFKLRGAYAMMAGLTEEQKAHGVITASAGNHAQGVAFSSARLGVKALIVMPTATADIKVDAVRGFGGEVLLHGANFDEAKAKAIELSQQQGFTWVPPFDHPMVIAGQGTLALELLQQDAHLDRVFVPVGGGGLAAGVAVLIKQLMPQIKVIAVEAEDSACLKAALDAGHPVDLPRVGLFAEGVAVKRIGDETFRLCQEYLDDIITVDSDAICAAMKDLFEDVRAVAKPSGALALAGMKKYIALHNIRGERLAHILSGANVNFHGLRYVSERCELGEQREALLAVTIPEEKGSFLKFCQLLGGRSVTEFNYRFADAKNACIFVGVRLSRGLEERKEILQMLNDGGYSVVDLSDDEMAKLHVRYMVGGRPSHPLQERLYSFEFPESPGALLRFLNTLGTYWNISLFHYRSHGTDYGRVLAAFELGDHEPDFETRLNELGYDCHDETNNPAFRFFLAG

SEQ ID NO: 9 - DNA, 1548 bp, Escherichia coli
  CDS (527)..(952): yjgF-Orf
tcgcgatctggtactgtaaggggaaatagagatgacacacgataataaattgcaggttgaagctattaaacgcggcacggtaattgaccatatccccgcccagatcggttttaagctgttgagtctgttcaagctgaccgaaacggatcagcgcatcaccattggtctgaacctgccttctggcgagatgggccgcaaagatctgatcaaaatcgaaaatacctttttgagtgaagatcaagtagatcaactggcattgtatgcgccgcaagccacggttaaccgtatcgacaactatgaagtggtgggtaaatcgcgcccaagtctgccggagcgcatcgacaatgtgctggtctgcccgaacagcaactgtatcagccatgccgaaccggtttcatccagctttgccgtgcgaaaacgcgccaatgatatcgcgctcaaatgcaaatactgtgaaaaagagttttcccataatgtggtgctggccaattaattgcggttggtaataaaagtctggctccctataatgagccagactttttaccgctgtaataaaggagaaatcatgagcaaaactatcgcgacggaaaatgcaccggcagctatcggtccttacgtacagggcgttgatctgggcaatatgatcatcacctccggtcagatcccggtaaatccgaaaacgggcgaagtaccggcagacgtcgctgcacaggcacgtcagtcgctggataacgtaaaagcgatcgtcgaagccgctggcctgaaagtgggcgacatcgttaaaactaccgtgtttgtaaaagatctgaacgacttcgcaaccgtaaacgccacttacgaagccttcttcaccgaacacaacgccaccttcccggcacgttcttgcgttgaagttgcccgtctgccgaaagacgtgaagattgagatcgaagcgatcgctgttcgtcgctaatcttgatggaaatccgggctatcatgcccggattaagtctgatgacaaacgcaaaatcgcctgatgcgctacgcttatcaggcctacgtgattcctgcaatttattgaatttgttggccggataaggcatttacgccgcatccggcatgaacaaaactcactttgtctacaatctgaatcggggctatcgtgcccagtttattctttattgccagccgtaacgacggctatagaaccctttcaccaactgggttaatgtcatataccctgccagaatcgcaaccagccacgggaaatagcttaacggcagcgcctgtaattgcagataactggccagcggtgaaaacggcaatgcgatcccgacaatcatcacgatcacggtcatgatcattaacggccacgatgcacagctctgaataaacggcacacggcgggtgcggatcatatgcacaatcagcgtttgcgacagtaagcccaccacaaaccatcccgactggaacagcgtttgcgtttccggcgtgttggcatggaatacccaccacatcaggcaaaacgtcaaaatatcgaagatcgagctgatcggtccgaagaagatc

SEQ ID NO: 10 - protein, 141 aa, Escherichia coli
MSQTFYRCNKGEIMSKTIATENAPAAIGPYVQGVDLGNMIITSGQIPVNPKTGEVPADVAAQARQSLDNVKAIVEAAGLKVGDIVKTTVFVKDLNDFATVNATYEAFFTEHNATFPARSCVEVARLPKDVKIEIEAIAVRR

(19) United States
(12) Patent Application Publication
(10) Pub. No.: US 2007/0092950 A1
(43) Pub. Date: Apr. 26, 2007

(54) Process for preparing l-threonine

(75) Inventors: Daniela Kruse, Bielefeld (DE); Thomas Hermann, Bielefeld (DE); Georg Thierbach, Bielefeld (DE); Mechthild Rieping, Bielefeld (DE)

Correspondence Address: FITCH, EVEN, TABIN & FLANNERY, P. O. BOX 18415, WASHINGTON, DC, 20036, US

(73) Assignee: Degussa AG, Dusseldorf (DE)

(21) Appl. No.: 10/567,749

(22) PCT Filed: Jul. 29, 2004
(86) PCT No.: PCT/EP04/08470

§ 371 Date: Feb. 10, 2006

Related U.S. Application Data
(60) Provisional application No. 60/494,566, filed on Aug. 13, 2003.

(30) Foreign Application Priority Data
Aug. 12, 2003 (DE) 103 37 028.5
Jun. 18, 2004 (DE) 10 2004 029 639.1

(51) Int. Cl. C12P 13/04; C12N 1/21

(52) U.S. Cl. 435/106; 435/252.33

(57) ABSTRACT

The invention provides an improved process for the fermentative preparation of L-threonine using L-threonine-producing bacteria from the family Enterobacteriaceae.

### CROSS REFERENCE TO RELATED APPLICATIONS

The present application represents U.S. national stage of-international application PCT/EP2004/008470, which had an international filing date of Jul. 29, 2004, and which was published in English under PCT Article 21(2) on Feb. 17, 2005. The international application claims priority to German applications 103 37 028.5, filed on Aug. 13, 2003; and 10 2004 029 639.1, filed on Jun. 18, 2004. The international application also claims priority to U.S. provisional application No. 60/494,566, filed on Aug. 13, 2003. These prior applications are hereby incorporated by reference in their entirety.

### FIELD OF THE INVENTION

The invention provides an improved process for the fermentative preparation of L-threonine using bacteria from the family Enterobacteriaceae.

### BACKGROUND OF THE INVENTION

L-threonine is used in human medicine, in the pharmaceutical industry, in the foodstuffs industry and very particularly in animal nutrition.

It is known that L-threonine can be prepared by fermentation from strains of the family Enterobacteriaceae, in particular Escherichia coli. Due to the great importance of this amino acid, efforts are constantly made to improve the method of preparation. Process improvements may be based on fermentation technology steps, such as e.g. stirring and supplying with oxygen, or the composition of the nutrient medium, such as e.g. the sugar concentration during fermentation, or working up to give the final product by e.g. ion exchange chromatography or the intrinsic, i.e. genetically based, performance characteristics of the bacterium itself.

U.S. Pat. No. 5,538,873 and EP-B-0593792 or Okamoto et al. (Bioscience, Biotechnology, and Biochemistry 61 (11), 1877-1882, 1997) describe how threonine can be prepared by fermentation in a batch process or a fed batch process. Furthermore, U.S. Pat. No. 6,562,601 describes a process for preparing L-threonine using strains of the family Enterobacteriaceae in which, after performing fermentation in a fed batch process, the fermentation broth is drained down to 1-90 vol. %, then the remaining fermentation broth is topped up with growth medium and, preferably after a growth phase, a further fermentation step is performed by the fed batch process mentioned. This process may be repeated several times and is therefore called a repeated fed batch process.

Another process for preparing threonine using bacteria from the family Enterobacteriaceae, in particular Escherichia coli, is described in the patent U.S. Pat. No. 6,562,601. This comprises first cultivating the bacterium in a fed batch process, wherein threonine is enriched in the fermentation broth. At a desired time, some, i.e. 10 to 99% of the fermentation broth present in the fermenter, is harvested. The remainder of the fermentation broth remains in the fermenter. The fermentation broth remaining in the fermenter is topped up with nutrient medium and another fermentation is performed using the fed batch process. The cycle described is optionally performed several times.

### OBJECT OF THE INVENTION

The object of the invention is to provide new measures for the improved fermentative preparation of L-threonine.

### SUMMARY OF THE INVENTION

The invention provides a fermentation process, characterized in that
a) the bacterium is inoculated into at least a first nutrient medium and cultivated, then
b) some of the fermentation broth is abstracted, wherein more than 90 vol. %, in particular more than 91 vol. %, more than 92 vol. %, more than 93 vol. %, more than 94 vol. %, more than 95 vol. %, more than 96 vol. %, more than 97 vol. % or more than 98 vol. % of the total volume of fermentation broth remains in the fermentation container and wherein a maximum of 99 vol. %, 99.3 vol. %, 99.6 vol. % or 99.9 vol. % of the total volume of the fermentation broth remains in the fermentation container, then
c) the remaining fermentation broth is topped up with one or more further nutrient media, wherein the further nutrient medium or further nutrient media contains at least one source of carbon, at least one source of nitrogen and at least one source of phosphorus, and cultivation is continued under conditions which enable the formation of L-threonine,
d) steps b) and c) are optionally performed several times, and
e) the concentration of the source(s) of carbon during cultivation in accordance with step c) and/or d) is adjusted to a maximum of 30 g/l.

### DETAILED DESCRIPTION OF THE INVENTION

Cultivation of the bacterium in accordance with step a) is performed typically in a fermenter (bioreactor). These have a volume of about 10-500 m³ (cubic meters) on an industrial production scale. On a laboratory scale, when the process according to the invention can be checked in a simple manner, fermenter volumes of 1-50 1 are typical. Fermenter volumes of 50 1 to 10 m³ are normally used on a pilot-plant scale.

The expression plant performance is understood to mean that the weight or amount of a product is produced with a certain yield and at a certain rate or with a certain productivity or space-time yield in a plant such as e.g. a fermenter. These parameters largely determine the cost or economic viability of a process.

A fermentation broth is understood to be the suspension of a microorganism being produced by the cultivation of a microorganism, in the case of the present invention a L-threonine-producing bacterium, in a nutrient medium using a fermenter.

According to the invention, the plant performance of a L-threonine-producing fermenter can be increased by cultivating by the batch process or the fed batch process in the first step a) described above, wherein when using the fed batch process at least one additional nutrient medium is used. In step b) described above, the culture fermentation broth is withdrawn, wherein less than 10 vol. %, in particular less than 9 vol. %, less than 8 vol. %, less than 7 vol. %, less than 6 vol. %, less than 5 vol. %, less than 4 vol. %, less than 3 vol. %, less than 2 vol. % of the total volume of the fermentation broth is abstracted, and wherein a minimum of 1 vol. %, 0.7 vol. %, 0.4 vol. % or 0.1 vol. % of the total volume of the fermentation broth is abstracted. Accordingly, more than 90 up to a maximum of 99.9 vol. % of the fermentation broth remains in the fermenter in the process according to the invention, in accordance with step b).

Then, in step c) the remaining fermentation broth is topped up with one or more further nutrient media, up to about 100% of the original volume, wherein the further nutrient medium or further nutrient media contains at least one source of carbon, at least one source of nitrogen and at least one source of phosphorus, and cultivation continues under conditions which enable the formation of L-threonine. This step c) is optionally repeated several times. The L-threonine formed is collected and optionally purified and isolated.

During cultivation step a), the bacterium is inoculated into at least a first nutrient medium and is cultivated by the batch process or the fed batch process. When using the fed batch process, an added nutrient medium is supplied after more than 0 up to a maximum of 10 hours, advantageously after 1 to 10 hours, preferably after 2 to 10 hours and particularly preferably after 3 to 7 hours.

The first nutrient medium contains, as a source of carbon, one or more compounds chosen from the group saccharose, molasses from sugar beet or sugar cane, fructose, glucose, starch hydrolysate, lactose, galactose, maltose, xylose, cellulose hydrolysate, arabinose, acetic acid, ethanol and methanol in concentrations of 1 to 100 g/kg or 1 to 50 g/kg, preferably 10 to 45 g/kg, particularly preferably 20 to 40 g/kg. Starch hydrolysate is understood to mean the hydrolysate from corn, cereals, potatoes or tapioca.

Sources of nitrogen which can be used in the first nutrient medium may be organic nitrogen-containing compounds such as peptones, yeast extract, meat extract, malt extract, corn steep liquor, soy bean flour and urea or inorganic compounds such as ammonia, ammonium sulfate, ammonium chloride, ammonium phosphate, ammonium carbonate and ammonium nitrate, potassium nitrate, potassium sodium nitrate. The sources of nitrogen may be used individually or as a mixture in concentrations of 1 to 40 g/kg, preferably 1 to 30 g/kg or 10 to 30 g/kg, particularly preferably 1 to 25 g/kg or 10 to 25 g/kg, very particularly preferably 1 to 30 g/kg or 1 to 25 g/kg.

Sources of phosphorus which may be used in the first nutrient medium are phosphoric acid, alkali metal or alkaline earth metal salts of phosphoric acid, in particular potassium dihydrogen phosphate or dipotassium hydrogen phosphate or the corresponding sodium-containing salts, polymers of phosphoric acid or the hexaphosphate of inositol, also called phytic acid, or the alkali metal or alkaline earth metal salts thereof in concentrations of 0.1 to 5 g/kg, preferably 0.3 to 3 g/kg, particularly preferably 0.5 to 1.5 g/kg. The first nutrient medium must also contain salts of metals, such as e.g. magnesium sulfate or iron sulfate, which are required for growth. These substances are present in concentrations of 0.003 to 3 g/kg. Finally, essential growth substances such as amino acids (e.g. homoserine) and vitamins (e.g. thiamine) are used in addition to the substances mentioned above. Antifoaming agents, such as e.g. polyglycol esters of fatty acids, may also used to control the production of foam.

The added nutrient medium which is used in a fed batch process generally contains, simply as a source of carbon, one or more of the compounds chosen from the group saccharose, molasses from sugar beet or sugar cane, fructose, glucose, starch hydrolysate, lactose, galactose, maltose, xylose, cellulose hydrolysate, arabinose, acetic acid, ethanol and methanol in concentrations of 300 to 700 g/kg, preferably 400 to 650 g/kg, and optionally an inorganic source of nitrogen such as e.g. ammonia, ammonium sulfate, ammonium chloride, ammonium phosphate, ammonium carbonate, ammonium nitrate, potassium nitrate or potassium sodium nitrate. Alternatively, these and other components may also be fed separately.

It was found that in the process according to the invention, in accordance with step c) and/or d), the constituents of the further nutrient medium may be supplied to the culture in the form of a single further nutrient medium as well as in a number of further nutrient media. According to the invention, the further nutrient medium is or the further nutrient media are supplied to the culture in at least one (1) feed stream or in a number of feed streams in least 2 to 10, preferably 2 to 7 or 2 to 5 feed streams.

The further nutrient medium or the further nutrient media contain(s), as a source of carbon, one or more compounds chosen from the group saccharose, molasses from sugar beet or sugar cane, fructose, glucose, starch hydrolysate, maltose, xylose, cellulose hydrolysate, arabinose, acetic acid, ethanol and methanol in concentrations of 20 to 700 g/kg, preferably 50 to 650 g/kg.

Furthermore, the further nutrient medium contains or the further nutrient media contain a source of nitrogen consisting of organic nitrogen-containing compounds such as peptones, yeast extract, meat extract, malt extract, corn steep liquor, soy bean flour and urea or inorganic compounds such as ammonia, ammonium sulfate, ammonium chloride, ammonium phosphate, ammonium carbonate, ammonium nitrate and/or potassium nitrate or potassium sodium nitrate. The sources of nitrogen may be used individually or as a mixture in concentrations of 5 to 50 g/kg, preferably 10 to 40 g/kg.

Furthermore, the further nutrient medium contains or the further nutrient media contain a source of phosphorus consisting of phosphoric acid or the alkali metal or alkaline earth metal salts of phosphoric acid, in particular potassium dihydrogen phosphate or dipotassium hydrogen phosphate or the corresponding sodium-containing salts, polymers of phosphoric acid or the hexaphosphate of inositol, also known as phytic acid, or the corresponding alkali metal or alkaline earth metal salts. The sources of phosphorus may be used individually or as a mixture in concentrations of 0.3 to 3 g/kg, preferably 0.5 to 2 g/kg. The further nutrient medium or further nutrient media must also contain salts of metals, such as e.g. magnesium sulfate or iron sulfate, which are required for growth, in concentrations of 0.003 to 3 g/kg, preferably in concentrations of 0.008 to 2 g/kg. Finally, essential growth substances such as amino acids (e.g. homoserine) and vitamins (e.g. thiamine) are used in addition to the substances mentioned above. Antifoaming agents, such as e.g. polyglycol esters of fatty acids, may also used to control the production of foam.

When using a single further nutrient medium, this is typically supplied to the culture in one feed stream. When using a number of further nutrient media, these are supplied in a corresponding number of feed streams. When using a number of further nutrient media, it should be noted that each of these may contain only one of the sources of carbon, nitrogen or phosphorus mentioned, or else a mixture of the sources of carbon, nitrogen or phosphorus mentioned.

According to the invention, the fed further nutrient medium or the fed further nutrient media is adjusted in such a way that a phosphorus to carbon ratio (P/C ratio) of at most 4; of at most 3; of at most 2; of at most 1.5; of at most 1; of at most 0.7; of at most 0.5; at most 0.48; at most 0.46; at most 0.44; at most 0.42; at most 0.40; at most 0.38; at most 0.36; at most 0.34; at most 0.32; at most 0.30 mmoles of phosphorus per mole of carbon is present.

The abstraction of fermentation broth described in step b) takes place in less than 180 minutes, preferably in less than 120 minutes, particularly preferably in less than 60 minutes and very particularly preferably in less than 30 to less than 15 minutes.

If a further nutrient medium or several further nutrient media are used for topping up as described in step c), this topping up may take place in the form of one or several batches or feedstocks or continuously or using a combination of the two procedures. A final top-up level of about 100% of the original volume is again reached. The expression “about 100%” in this context means that some variations may occur within the scope of the technical possibilities which may lead to the final top-up level being, for example, 97%-103%, 98%-102%, 99-101%, 99.5-100.5% or 99.9-100.1% of the original volume.

If topping up takes place in the form of one or several batches, this occurs, according to the invention, as rapidly as possible i.e. in less than 180 minutes, preferably in less than 120 minutes, particularly preferably in less than 60 minutes, particularly preferably in less than 30 minutes to less than 15 minutes. After topping-up to about 100% of the original volume as described above, cultivation takes place until the source of carbon has been consumed or up to another suitable time shortly before complete consumption of the source of carbon, before again abstracting fermentation broth in accordance with step b). At this point, the concentration of the source of carbon is >0 to ≦5 g/l, >0 to ≦3 g/l, >0 to ≦2 g/l, >0 to ≦1 g/l, >0 to ≦0.5 g/l.

During a continuous topping up procedure, then topping up with one or more further nutrients takes place until approximately 100% of the original volume is reached again. The fermentation broth is then cultivated further until the source of carbon has been consumed or almost (see above) consumed.

When using a combination of the two procedures one or more further nutrient media in the form of one or more batches are added as rapidly as possible and then one or more further nutrient media are introduced continuously with continuing cultivation. The fermentation broth is cultivated further until the source of carbon has been consumed or almost (see above) consumed.

Cultivation in steps a) and c) is performed under conditions which enable the formation of L-threonine. During cultivation the temperature is adjusted to be within the range 27 to 45° C., preferably 29 to 42° C., particularly preferably 33 to 40° C. Fermentation can be performed at atmospheric pressure or optionally under an excess pressure, preferably at 0 to 2.5 bar excess pressure, particularly preferably at 0 to 1.5 bar. The oxygen partial pressure is regulated to 5 to 50%, preferably about 20%, of the saturation value for air.

Controlling the pH to a value of about 6 to 8, preferably 6.5 to 7.5 can be performed with 25% strength ammonia water. The conditions for cultivation may remain constant or may alter during cultivation. Likewise, the cultivation conditions in steps a) and c) may be identical or different.

Repeating steps b) and c) in accordance with d) takes place >(greater than) 0 to 100 times, preferably 2 to 90 or 2 to 80 times, particularly preferably 4 to 70, 4 to 60, 4 to 50 or 4 to 40 times and particularly preferably 5 to 30, 6 to 30, 7 to 30, 8 to 30, 9 to 30 or 10 to 30 times.

The time between abstracting at least 0.1 vol. % to less than 10 vol. % of the total volume of fermentation broth, complete topping up to about 100%, subsequent cultivation and renewed abstraction of the fermentation broth is at most 10 hours or at most 5 hours, preferably at most 3 hours, particularly preferably at most 2 hours to at most 1 hour.

Accordingly, abstraction of the fermentation broth, topping up with nutrient medium, subsequent cultivation and renewed abstraction of fermentation broth takes place at a rate which corresponds to an average residence time of less than 100 hours or less than 50 hours, preferably less than 30, very particularly preferably less than 20 or less than 10 hours. The average residence time is the theoretical time that the particles remain within a culture. The average residence time is described by the ratio of the volume of liquid in the reactor to the amount which flows through (Biotechnologie; H. Weide, J. Páca and W. A. Knorre; Gustav Fischer Verlag Jena; 1991). The amount which flows through is defined by the volume of fermentation broth drained off or the volume of nutrient medium or further nutrient media used for topping up. Measurement of the full status can be performed directly, e.g. using a radar measurement, or indirectly, e.g. using a weight determination.

According to the invention, the concentration of the source of carbon during cultivation in accordance with step c) and/or d) is adjusted in general to at most 30 g/l, to at most 20 g/l, to at most 10 g/l, preferably to at most 5 g/l, particularly preferably at most 2 g/l. This concentration is held steady for at least 75%, preferably for at least 85%, particularly preferably for at least 95% of the time of cultivation in accordance with step b) and/or c). The concentration of the source of carbon is determined using methods which are disclosed in the prior art. β-D-glucose is determined, for example, in a glucose analyzer, YSI 02700 Select, from Yellow Springs Instruments (Yellow Springs, Ohio, USA).

Optionally, the withdrawn culture broth can be provided with oxygen or an oxygen-containing gas, optionally with stirring, until the concentration of the source of carbon falls to below 2 g/l; below 1 g/l; or below 0.5 g/l.

In a process according to the invention, the yield is at least 31%; at least 33%; at least 35%; at least 37%; at least 40%, at least 42%; at least 44%; at least 46%; at least 48%. Here, the yield is defined as the ratio of the total amount of L-threonine formed in a cultivation process to the total amount of the source of carbon used or consumed.

In a process according to the invention, L-threonine is formed with a space-time yield of at least 1.5 to 2.5 g/l per hr., at least 2.5 to 3.5 g/l per hr., at least 2.5 to more than 3.5 g/l per hr., at least 3.5 to 5.0 g/l per hr., at least 3.5 to more than 5.0 g/l per hr., or at least 5.0 to 8.0 g/l or more per hr. The space-time yield is defined as the ratio of the total amount of threonine formed in a cultivation process to the volume of the culture, regarded over the entire time of cultivation. The space-time yield is also known as the volumetric productivity.

Naturally, in a fermentation process like the one according to the invention, the product is produced with a certain yield and with a certain space-time yield (volumetric productivity). In a process according to the invention, L-threonine can be produced with a yield of at least 31% and a space-time yield of at least 1.5 to 2.0 g/l per hour. Further couplings of yield and space-time yield such as for example a yield of at least 37% and a space-time yield of at least 2.5 g/l per hour are easily produced from the specifications given above.

L-threonine can be recovered, collected or concentrated from the withdrawn culture broth and optionally purified.

It is also possible to produce a product from the withdrawn culture broth (=fermentation broth) by removing the biomass of bacterium present in the culture broth completely (100%) or almost completely i.e. by removing more than or greater than (>) 90%, 95%, 97%, 99% of the biomass and largely leaving behind the other constituents of the fermentation broth, i.e. leaving 30%-100%, 40%-100%, 50%-100%, 60%-100%, 70%-100%, 80%-100% or 90%-100% of these, preferably greater than or equal to (≧) 50%, ≧60%, ≧70%, ≧80%, ≧90% or ≧95% of these or even the entire amount (100%) of these in the product.

Separation methods such as for example centrifuging, filtering, decanting, flocculating or a combination of these are used to remove or isolate the biomass.

The broth obtained is then thickened or concentrated using known methods such as for example by using a rotary evaporator, thin layer evaporator or falling film evaporator, by reverse osmosis, by nanofiltration or by a combination of these.

This concentrated broth is then processed using the methods of freeze-drying, spray-drying, spray granulation or any other process to give a preferably free flowing, finely divided powder. This free-flowing finely divided powder can then again be converted into a coarse-grained, very free-flowing, storable and largely dust-free product by using suitable compacting or granulating processes. Altogether, more than 90% of the water is removed in this way so that the water content of the product is less than 10%, less than 5%.

The process steps mentioned above do not necessarily have to be performed in the sequence specified here, but they may optionally be combined in a technically meaningful manner.

The process according to the invention is characterized in particular by an increased space-time yield when compared with a conventional fed batch process.

Analysis of L-threonine and other amino acids may be performed by anion exchange chromatography followed by ninhydrin derivation as described in Spackman et al. (Analytical Chemistry 30: 1190-1206 (1958)) or by reversed phase HPLC as described in Lindroth et al. (Analytical Chemistry 51: 1167-1174 (1979)).

To perform the process according to the invention, L-threonine-producing bacteria from the family Enterobacteriaceae, chosen from the genera Escherichia, Erwinia, Providencia and Serratia are suitable. The genera Escherichia and Serratia are preferred. From the genus Escherichia the species Escherichia coli is mentioned in particular and from the genus Serratia the species Serratia marcescens is mentioned in particular.

The bacteria contain at least one copy of a thrA gene or allele which codes for a threonine-insensitive aspartate kinase I—homoserine dehydrogenase I. In this connection, the literature mentions “feed back” resistant or even desensitized variants. These types of bacteria are typically resistant to the threonine analog α-amino-β-hydroxyvaleric acid (AHV) (Shiio and Nakamori, Agricultural and Biological Chemistry 33 (8), 1152-1160 (1969)). Biochemical tests relating to “feed back” resistant aspartate kinase I—homoserine dehydrogenase I variants are described for example in Cohen et al. (Biochemical and Biophysical Research Communications 19(4), 546-550 (1965)) and in Omori et al. (Journal of Bacteriology 175(3), 785-794 (1993)). Optionally, the threonine-insensitive aspartate kinase I—homoserine dehydrogenase I is overexpressed.

Methods of overexpression are adequately described in the prior art, for example in Makrides et al. (Microbiological Reviews 60 (3), 512-538 (1996)). The copy number is raised by at least one (1) copy by using vectors. Plasmids such as for example those described in U.S. Pat. No. 5,538,873 can be used as vectors. Phages, for example the phage Mu, as described in EP 0 332 448, or the phage lambda (λ) can also be used as vectors. An increase in the copy number can also be produced by incorporating a further copy at another site on the chromosome, for example at the att site on the phage λ (Yu and Court, Gene 223, 77-81 (1998)). U.S. Pat. No. 5,939,307 describes how an increase in expression can be produced by incorporating expression cassettes or promoters such as for example the tac promoter, trp promoter, lpp promoter or P_{L} promoter and P_{R} promoter upstream of the phage λ in the chromosomal threonine operon. Promoters in the phage T7, gear-box promoters or the nar promoter can also be used in the same way. These types of expression cassettes or promoters can also be used by overexpressing plasmid-bonded genes, as described in EP 0 593 792. There again, the expression of plasmid-bonded genes can be regulated by using the lacI^{Q} allele (Glascock and Weickert, Gene 223, 221-231 (1998)). Overexpression can also be produced by removing the attenuator in the threonine operon (Park et al., Biotechnology Letters 24, 1815-1819 (2002)) or by using the thr79-20 mutation (Gardner, Proceedings of the National Academy of Sciences, USA 76(4), 1706-1710 (1979)) or by mutation of the thrS gene coding for threonyl-t-RNA synthetase as described in Johnson et al. (Journal of Bacteriology 129(1), 66-70 (1977)). Using the measures described, the intracellular concentration of the particular aspartate kinase I—homoserine dehydrogenase I protein variants is increased by at least 10% as compared with the starting strain.

A suitable thrA allele is described in U.S. Pat No. 4,278,765 and is obtainable in the form of the strain MG442 from the Russian National Collection of Industrial Microorganisms (VKPM, Moscow, Russia) under accession number CMIM B-1628. Other suitable thrA alleles are described in WO 00/09660 and WO 00/09661 and are obtainable from the Korean Culture Centre for Microorganisms (KCCM, Seoul, Korea) under accession numbers KCCM 10132 and KCCM 10133. Another suitable thrA allele is present in the strain H-4581, which is described in U.S. Pat. No. 4,996,147 and is obtainable under accession number Ferm BP-1411 from the National Institute of Advanced Industrial Science and Technology (1-1-1 Higashi, Tsukuba Ibaraki, Japan). Finally, further thrA alleles are described in U.S. Pat. No. 3,580,810 and these are obtainable in the form of strains ATCC 21277 and ATCC 21278 deposited at ATCC. Another allele is described in U.S. Pat. No. 3,622,453 and is obtainable from ATCC in the form of strain KY8284, under accession number ATCC 21272. In addition, another thrA allele is described in WO 02/064808 and is deposited at KCCM in the form of strain pGmTN-PPC12, under accession number KCCM 10236.

Optionally, thrA alleles which code for “feed back” resistant aspartate kinase I—homoserine dehydrogenase I variants can be isolated using the adequately well-known methods of mutagenesis of cells using mutagenic substances, for example N-methyl-N′-nitro-N-nitroso-guanidine (MNNG) or ethylmethane sulfonate (EMS) or mutagenic radiation, for example UV radiation followed by selection of threonine analog (for example AHV) resistant variants. These types of mutagenesis methods are described, for example, in Shiio and Nakamori (Agricultural and Biological Chemistry 33 (8), 1152-1160 (1969)) or in Saint-Girons and Margerita (Molecular and General Genetics 162, 101-107 (1978)) or in the well-known manual by J. H. Miller (A Short Course in Bacterial Genetics. A Laboratory Manual and Handbook for Escherichia coli and Related Bacteria, Cold Spring Harbor Laboratory Press, New York, USA, 1992) in particular on pages 135 to 156. Shiio and Nakamori, for example, treat a cell suspension of Escherichia coli with 0.5 mg/ml of MNNG in a 0.1 M sodium phosphate buffer at pH 7 for about 15 minutes at room temperature (i.e. in general at about 16 to 26° C.) to produce mutations. Miller recommends, for example, treating for 5 to 60 minutes with 30 μl EMS per 2 ml of cell suspension in 0.1 M Tris buffer at pH 7.5 at a temperature of 37° C. These mutagenesis conditions may be modified in an obvious manner. The selection of AHV-resistant mutants takes place on minimal agar which typically contains 2 to 10 mM AHV. The corresponding alleles may then be cloned and subjected to a sequence determination and the protein variants coded by these alleles subjected to an activity determination. Optionally, the mutants produced may also be used directly. The word “directly” means that the mutants produced can be used for the production of L-threonine in a process according to the invention or that further modifications to increase the performance characteristics of these mutants, such as for example attenuating threonine-degradation or overexpression of the threonine operon, may be performed.

In the same way, the methods of in vitro mutagenesis may also be used, as described, for example, in the well-known manual by Sambrook et al. (Molecular Cloning, A Laboratory Manual, 2nd ed., Cold Spring Harbor Laboratory Press, Cold Spring Harbor, N.Y., USA, 1989). Corresponding methods are also commercially available in the form of so-called “kits” such as, for example, the “QuikChange Site-Directed Mutagenesis Kit” supplied by Stratagene (La Jolla, USA) and described by Papworth et al. (Strategies 9(3), 3-4 (1996)).

These mutagenesis methods may naturally also be applied to other genes, alleles or strains or problems and tasks such as, for example, the production and isolation of mutants which are resistant to L-threonine.

Preferred thrA alleles are those which code for aspartate kinase I—homoserine dehydrogenase I variants which have at least 40%, at least 45%, at least 50%, at least 55% or at least 60% of the homoserine dehydrogenase activity in the presence of 10 mM of L-threonine and/or which have at least 70%, at least 75% or at least 80% of the homoserine dehydrogenase activity in the presence of 1 mM of L-threonine, in comparison to the activity in the absence of L-threonine. Optionally, the aspartate kinase activity of the aspartate kinase I—homoserine dehydrogenase I variants in the presence of 10 mM of L-threonine is at least 60%, at least 65%, at least 70%, at least 75% or at least 80% of the activity in the absence of L-threonine.

In addition, bacteria from the family Enterobacteriaceae which contain a stop codon chosen from the group opal, ochre and amber, preferably amber, in the rpoS gene and a t-RNA suppressor chosen from the group opal suppressor, ochre suppressor and amber suppressor, preferably amber suppressor, are suitable. The amber mutation is preferably at position 33 corresponding to the amino acid sequence of the RpoS gene product. supE is preferably used as amber suppressor. These bacteria are described in PCT/EP02/02055. A strain which contains the described mutation in the rpoS gene and the suppressor supE is obtainable, under accession number DSM 15189, from the German Collection of Microorganisms and Cell Cultures (Braunschweig, Germany).

The nucleotide sequence of the rpoS gene can be found in the prior art. The nucleotide sequence of the rpoS gene corresponding to accession number AE000358 is given as SEQ ID NO. 1. The amino acid sequence of the associated RpoS gene product or protein is given in SEQ ID NO. 2. The nucleotide sequence of a rpoS allele which contains a stop codon of the amber type at the site in the nucleotide sequence corresponding to position 33 of the amino acid sequence of the RpoS gene product or protein, corresponding to SEQ ID NO. 1 or SEQ ID NO. 2, is reproduced in SEQ ID NO. 3. The suppressor supE is described in the prior art and is given as SEQ ID NO. 4.

In addition, suitable bacteria from the family Enterobaceteriaceae are those which are not able to degrade threonine under aerobic culture conditions nor to use it as a source of nitrogen. Aerobic culture conditions are understood to be those in which the oxygen partial pressure in the fermentation culture,is greater than (>) 0%, for 90%, preferably 95%, very particularly preferably 99% of the fermentation time. A strain of this type is, for example, the strain KY10935 described by Okamoto (Bioscience, Biotechnology and Biochemistry 61(11), 1877-1882 (1997)). Strains which are not able to degrade threonine with the elimination of nitrogen generally have an attenuated threonine dehydrogenase (EC 1.1.1.103) coded by the tdh gene. The enzyme was described by Aronson et al. (The Journal of Biological Chemistry 264(9), 5226-5232 (1989)). Attenuated tdh genes are described, for example, in Ravnikar and Somerville (Journal of Bacteriology, 1986, 168(1), 434-436) in U.S. Pat. No. 5,705,371, in WO 02/26993 and in Komatsubara (Bioprocess Technology 19, 467-484 (1994)).

A suitable tdh allele is described in U.S. Pat. No. 5,538,873 and is obtainable, in the form of strain B-3996 under accession number 1876, from the Russian National Collection of Industrial Microorganisms (VKPM, Moscow, Russia). Another tdh allele is described in U.S. Pat. No. 5,939,307 and is obtainable in the form of strain kat-13 under accession number NRRL B-21593, from the Agricultural Research Service Patent Culture Collection (Peoria, Ill., USA). Finally, a tdh allele is described in WO 02/26993 and is deposited at NRRL in the form of strain TH21.97, under accession number NRRL B-30318. The allele tdh-1::cat1212 coding for a defective threonine dehydrogenase is obtainable from the E. coli Genetic Stock Centre (New Haven, Conn., USA) under accession number CGSC 6945.

In addition, bacteria from the family Enterobacteriaceae which possess an at least partial isoleucine requirement (“leaky” phenotype) which can be compensated for by the addition of L-isoleucine at a concentration of at least 10, 20 or 50 mg/l or L-threonine at a concentration of at least 50, 100 or 500 mg/l, are also suitable.

A requirement or auxotrophy is generally understood to mean that a strain has completely lost, for example, an enzyme activity, due to a mutation of a wild type function and requires the addition of a supplement, for example an amino acid, in order to grow. Partial requirement or partial auxotrophy is referred to when, for example, the activity of an enzyme from the biosynthetic pathway for an amino acid is impaired or attenuated but not completely switched off, due to a mutation of a wild type function. Strains with partial requirement typically have, in the absence of the supplement, a reduced, i.e. greater than (>) 0% and less than (<) 90%, 50%, 25% or 10%, rate of growth as compared to that of the wild type. In the literature, this connection is also called a “leaky” phenotype or “leakiness” (Griffiths et al.: An Introduction to Genetic Analysis, 6th edition, 1996, Freeman and Company, New York, USA).

A strain with this type of partial isoleucine requirement is described, for example, in WO 01/14525 and is deposited at KCCM in the form of strain DSM9906, under accession number KCCM 10168. Threonine-releasing or -producing strains with an isoleucine requirement generally have an attenuated threonine deaminase coded by the ilvA gene (E.C. number 4.3.1.19). Threonine deaminase is also known by the name threonine dehydratase. An attenuated ilvA gene which causes partial isoleucine auxotrophy is described, for example, in U.S. Pat. No. 4,278,765 and is obtainable from VKPM in the form of strain MG442, deposited under accession number B-1682.

Another attenuated ilvA gene is described, for example in WO 00/09660 and is obtainable from KCCM in the form of strain DSM 9807, deposited under accession number KCCM-10132. Further attenuated ilvA genes are described in Komatsubara (Bioprocess Technology 19, 467-484 (1994)).

The amino acid sequence of a suitable and new threonine deaminase comprises, for example, the sequence in SEQ ID NO. 6, wherein any amino acid except glutamic acid may be present at position 286. Glutamic acid is preferably replaced by lysine (E286K).

The expression “amino acid” is intended to mean in particular the proteinogenic L-amino acids, including the salts thereof, chosen from the group L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophane, L-proline and L-arginine.

SEQ ID NO. 8 gives the amino acid sequence of a threonine deaminase which contains the amino acid lysine at position 286; the associated nucleotide sequence is given as SEQ ID NO. 7. This contains the nucleobase adenine at position 856.

A different suitable threonine deaminase is the variant described by Lee et al. (Journal of Bacteriology 185 (18), 5442-5451 (2003)), in which serine at position 97 is replaced by phenylalanine (S97F). Further suitable threonine deaminases are the variants described by Fischer and Eisenstein (Journal of Bacteriology 175 (20), 6605-6613 (1993)), which possess at least one amino acid substituent chosen from the group: replacement of asparagine at position 46 by aspartic acid (N46D), replacement of alanine at position 66 by valine (A66V), replacement of proline at position 156 by serine (P156S), replacement of glycine at position 248 by cysteine (G248C) and replacement of aspartic acid at position 266 by tyrosine (D266Y).

By using insertion or deletion mutagenesis of at least one base pair or nucleotide or by insertion or deletion of at least one codon in the coding region or by incorporating a stop codon by transition or transversion mutagenesis in the coding region of the ilvA gene, alleles in which expression of the ilvA gene is generally completely switched off can be isolated. This method can also be transferred to other genes, alleles or open reading frames such as, for example, the tdh gene coding for threonine dehydrogenase.

In addition, suitable bacteria from the family Enterobacteriaceae are those which are resistant to inhibition by L-threonine and/or L-homoserine during growth. Threonine-resistant strains and the preparation thereof are described, for example, in Astaurova et al. (Prikladnaya Biokhimia Microbiologiya (1985), 21(5), 485 as English translation: Applied Biochemistry and Microbiology (1986), 21, 485-490)). The mutant described by Austaurova is resistant to 40 mg/ml of L-threonine. Furthermore, the strain 472T23, which can grow in the presence of 5 mg/ml of L-threonine and is at the same time resistant to L-homoserine, is described, for example, in U.S. Pat. No. 5,175,107. Strain 472T232 is obtainable from VKPM under accession number BKIIM B-2307 and from ATCC under the number ATCC 9801. Furthermore, WO 00/09660 describes strain DSM 9807 which can grow on a solid nutrient medium which contains 7% of L-threonine. Strain DSM 9807 is obtainable from KCCM under accession number KCCM-10132. Finally, WO 01/14525 describes strain DSM 9906 which can grow in a medium which contains 60% to 70% of a L-threonine fermentation mother liquor. Strain DSM 9906 is obtainable from KCCM under accession number KCCM-10168.

It is known (see EP 0994 190 A2 and Livshits et al. (Research in Microbiology 154, 123-135 (2003)), that resistance to L-threonine and L-homoserine is brought about by enhancing the rhtA gene. Enhancement can be produced by increasing the copy number of the gene or by using the rhtA23 mutation.

EP 0 994 190 A2 discloses that enhancement of the rhtB gene causes resistance to L-homoserine and L-threonine, in particular to L-homoserine, and improves threonine production. The minimum inhibition concentration of 250 μg/ml can be raised to 30000 μg/ml by overexpressing the RhtB gene product in a strain called N99.

EP 1 013 765 A1 discloses that enhancement of the rhtC gene brings about resistance to L-threonine and improves threonine production. A strain which is designated resistant to L-threonine is one which can grow in the presence of a concentration of at least 30 mg/ml of L-threonine on a minimal agar. Furthermore, it is disclosed that enhancement of the rhtB gene brings about resistance to L-homoserine and improves threonine production. A strain which is designated as resistant to L-homoserine is one which can grow in the presence of a concentration of at least 5 mg/ml of L-homoserine on a minimal agar. Strains are described in the patent application mentioned which are resistant to 10 mg/ml of L-homoserine and resistant to 50 mg/ml of L-threonine. U.S. Pat. No. 4,996,147 describes the strain H-4581 which is resistant to 15 g/l of homoserine. Strain H-4581 is obtainable from the National Institute of Advanced Industrial Science and Technology, under accession number FERM BP-1411.

EP 1 016 710 A2 discloses that enhancing the open reading frame or gene yfiK or yeaS brings about resistance to L-threonine and L-homoserine. The minimum inhibition concentration with respect to L-homoserine of 500 μg/ml can be increased to 1000 μg/ml and with respect to L-threonine can be increased from 30000 μg/ml to 40000 μg/ml by overexpressing the YfiK gene product in a strain called TG1. The minimum inhibition concentration with respect to L-homoserine of 500 μg/ml can be increased to 1000 μg/ml and with respect to L-threonine can be increased from 30000 μg/ml to 50000 μg/ml by overexpressing the YeaS gene product. Furthermore, it is shown, in the patent application mentioned, that threonine production can be improved by overexpressing the YfiK gene product.

In accordance with these technical instructions, strains were prepared which can grow in the presence of ≧ (at least) ≧5 g/l, ≧10 g/l, ≧20 g/l, ≧30 g/l, ≧40 g/l, ≧50 g/l, ≧60 g/l and ≧70 g/l of L-threonine, i.e. are resistant to L-threonine and are suitable for the production of L-threonine in a process according to the invention.

Strains which have at least the following features are particularly suitable for use in the process according to the invention:
a) a threonine-insensitive aspartate kinase I—homoserine dehydrogenase I, which is optionally present overexpressed, and
b) a stop codon chosen from the group opal, ochre and amber, preferably amber in the rpoS gene, and a t-RNA suppressor chosen from the group opal suppressor, ochre suppressor and amber suppressor, preferably amber suppressor.

In addition, strains which have at least the following features are particularly suitable for use in the process according to the invention:
a) a threonine-insensitive aspartate kinase I—homoserine dehydrogenase I, which is optionally present overexpressed,
b) are not able, under aerobic culture conditions, to degrade threonine, preferably due to the attenuation of threonine dehydrogenase,
c) an at least partial isoleucine requirement, and
d) can grow in the presence of at least 5 g/l of threonine.

Strains which have at least the following features are very particularly suitable for use in the process according to the invention:
a) a threonine-insensitive aspartate kinase I—homoserine dehydrogenase I, which is optionally present overexpressed,
b) a stop codon chosen from the group opal, ochre and amber, preferably amber in the rpoS gene, and a t-RNA suppressor chosen from the group opal suppressor, ochre suppressor and amber suppressor,
c) are not able, under aerobic culture conditions, to degrade threonine, preferably due to the attenuation of threonine dehydrogenase,
d) an at least partial isoleucine requirement, and
e) can grow in the presence of at least 5 g/l of threonine.

In addition, bacteria used for the process according to the invention may also have one or more of the following features:
attenuation of phosphoenolpyruvate-carboxykinase (PEP-carboxykinase)coded by the pckA gene as is described for example in WO 02/29080,
attenuation of phosphoglucose isomerase coded by the pgi gene (Froman et al. Molecular and General Genetics 217(1):126-31 (1989)).
attenuation of the YtfP gene product coded by open reading frame ytfp as is described for example in WO 02/29080,
attenuation of the YjfA gene product coded by open reading frame yjfA as is described for example in WO 02/29080,
attenuation of pyruvate oxidase coded by the poxB gene, as is described for example in WO 02/36797,
attenuation of the YjgF gene product coded by open reading frame yjgF as is described for example in PCT/EP03/14271. The yjgF Orf from Escherichia coli has been described by Wasinger VC. and Humphery-Smith I. (FEMS Microbiology Letters 169(2): 375-382 (1998)), Volz K. (Protein Science 8(11): 2428-2437 (1999)) and Parsons et al. (Biochemistry 42(1): 80-89 (2003)). The associated nucleotide and amino acid sequences are available in public data banks under accession number AE000495. For the sake of better clarity, these are given as SEQ ID NO. 9 and SEQ ID NO. 10.
enhancement of transhydrogenase coded by the genes pntA and pntB as is described for example in EP 0 733 712 A1,
enhancement of phosphoenolpyruvate synthase coded by the pps gene as is described for example in EP 0 877 090 A1,
enhancement of phosphoenolpyruvate carboxylase coded by the ppc gene as is described for example in EP 0 723 011 A1, and
enhancement of regulator RseB coded by the rseB gene as is described for example in EP 1382685. The regulator RseB has been described by Missiakas et al. (Molecular Microbiology 24(2), 355-371 (1997)), De Las Penas et al. (Molecular Microbiology 24(2): 373-385 (1997)) and Collinet et al. (Journal of Biological Chemistry 275(43): 33898-33904 (2000)). The associated nucleotide and amino acid sequences are available from public data banks under accession number AE000343.
enhancement of galactose-proton symporters (=galactose permease) coded by the galp gene as is described for example in DE 10314618.0. The galP gene and its function have been described by Macpherson et al. (The Journal of Biological Chemistry 258(7): 4390-4396 (1983)) and Venter et al. (The Biochemical Journal 363(Pt 2): 243-252 (2002)). The associated nucleotide and amino acid sequences are available from public data banks under accession number AE000377.
The ability to make use of saccharose as a source of carbon. Genetic determinants for the utilization of saccharose are described in the prior art, for example in FR-A-2559781, in Debabov (In: Proceedings of the IV International Symposium on Genetics of Industrial Microorganisms 1982. Kodansha Ltd, Tokyo, Japan, p 254-258), Smith and Parsell (Journal of General Microbiology 87, 129-140 (1975)) and Livshits et al. (In: Conference on Metabolic Bacterial Plasmids. Tartusk University Press, Tallin, Estonia (1982), p 132-134 and 144-146) and in U.S. Pat. No. 5,705,371. The genetic determinants for saccharose utilization of strain H155 described by Smith and Parsell were transferred by conjugation into a nalidixic acid-resistant mutant of Escherichia coli K-12 and the corresponding transconjugants deposited as DSM 16293 on the 16th Mar. 2004 at the German Collection of Microorganisms and Cell Cultures (Braunschweig, Germany). Genetic determinants for saccharose utilization are also present in the strain 472T23 described in U.S. Pat. No. 5,631,157 and this is obtainable from ATCC under the name ATCC 9801. Another genetic determinant for saccharose utilization was described by Bockmann et al. (Molecular and General Genetics 235, 22-32 (1992)) and is disclosed under the name csc system.
enhancement of the YedA gene product coded by open reading frame yeda as is described for example in WO 03/044191.
growth in the presence of at least 0.1 to 0.5 mM or at least 0.5 to 1 mM of borrelidin (borrelidin resistance) as is described in U.S. Pat. No. 5,939,307. Strain kat-13 which is resistant to borrelidin is obtainable from NRRL under accession number NRRL B-21593.
growth in the presence of at least 2 to 2.5 g/l or at least 2.5 to 3 g/l of diaminosuccinic acid (diaminosuccinic acid resistance) as described in WO 00/09661. The strain DSM 9806 which is resistant to diaminosuccinic acid is obtainable from KCCM under accession number KCCM-10133.
growth in the presence of at least 30 to 40 mM or at least 40 to 50 mM of α-methylserine (α-methylserine resistance) as described in WO 00/09661. Strain DSM 9806 which is resistant to α-methylserine is obtainable from KCCM under accession number KCCM-10133.
growth in the presence of at most 30 mM or at most 40 mM or at most 50 mM of fluoropyruvic acid (fluoropyruvic acid sensitivity) as described in WO 00/09661. The strain DSM 9806 which is sensitive to fluoropyruvic acid is obtainable from KCCM under accession number KCCM-10133.
growth in the presence of at least 210 mM or at least 240 mM or at least 270 mM or at least 300 mM of L-glutamic acid (glutamic acid resistance) as described in WO 00/09660. Strain DSM 9807 which is resistant to glutamic acid is obtainable from KCCM under accession number KCCM-10132.
an at least partial requirement for methionine. A strain with an at least partial methionine requirement is the strain H-4257 described in U.S. Pat. No. 5,017,483 and is obtainable from the National Institute of Advanced Industrial Science and Technology under accession number FERM BP-984. The requirement can be compensated by adding at least 25, 50 or 100 mg/l of L-methionine.
an at least partial requirement for m-diaminopimelic acid. A strain with an at least partial m-diaminopimelic acid requirement is the strain H-4257 described in U.S. Pat. No. 5,017,483 and this is obtainable from the National Institute of Advanced Industrial Science and Technology under accession number FERM BP-984. The requirement can be compensated by adding at least 25, 50 or 100 mg/l of m-diaminopimelic acid.
growth in the presence of at least 100 mg/l of rifampicin (rifampicin resistance) as described in U.S. Pat. No. 4,996,147. The strain H-4581 which is resistant to rifampicin is obtainable from the National Institute of Advanced Industrial Science and Technology under accession number FERM BP-1411.
growth in the presence of at least 15 g/l of L-lysine (lysine resistance) as described in U.S. Pat. No. 4,996,147. The strain H-4581 which is resistant to L-lysine is obtainable from the National Institute of Advanced Industrial Science and Technology under accession number FERM BP-1411.
growth in the presence of at least 15 g/l of methionine (methionine resistance) as described in U.S. Pat. No. 4,996,147. The strain H-4581 which is resistant to methionine is obtainable from the National Institute of Advanced Industrial Science and Technology under accession number FERM BP-1411.
growth in the presence of at least 15 g/l of L-aspartic acid (aspartic acid resistance) as described in U.S. Pat. No. 4,996,147. The strain H-4581 which is resistant to L-aspartic acid is obtainable from the National Institute of Advanced Industrial Science and Technology under accession number FERM BP-1411.
enhancement of pyruvate carboxylase coded by the pyc gene. Suitable pyc genes or alleles are, for example, those from Corynebacterium glutamicum (WO 99/18228, WO 00/39305 and WO 02/31158), Rhizobium etli (U.S. Pat. No. 6,455,284), Bacillus subtilis (EP 1092776). Optionally, the pyc gene from other microorganisms which contain an endogenous pyruvate carboxylase may also be used, such as for example Methanobacterium thermoautotrophicum or Pseudomonas fluorescens.

When using saccharose-containing nutrient media, the strains are provided with the genetic determinants for saccharose utilization.

The expression “enhancement” in this connection describes the increase in intracellular activity or concentration of one or more enzymes or proteins in a microorganism which are coded by the corresponding DNA by, for example, increasing the copy number of the open reading frame, gene or allele or open reading frames, genes or alleles by at least one (1) copy, by using a strong promoter or a gene or allele which codes for a corresponding enzyme or protein with high activity and optionally by combining these steps.

When using the measure of enhancement and also when using the measure of attenuation, the use of endogenous genes, alleles or open reading frames is generally preferred. “Endogenous genes” or “endogenous nucleotide sequences” are understood to be the genes or open reading frames or alleles and nucleotide sequences present in the population of a species.

When using plasmids to increase the copy number, these are optionally stabilized by one or more genetic loci chosen from the group comprising the parB locus of the plasmid R1 described by Rasmussen et al. (Molecular and General Genetics 209 (1), 122-128 (1987)), Gerdes et al. (Molecular Microbiology 4 (11), 1807-1818 (1990)) and Thistedt und Gerdes (Journal of Molecular Biology 223 (1), 41-54 (1992)), the flm locus of the F plasmid described by Loh et al. (Gene 66 (2), 259-268 (1988)), the par locus of the plasmid pSC101 described by Miller et al. (Gene 24 (2-3), 309-315 (1983), the cer locus of the plasmid ColE1 described by Leung et al. (DNA 4 (5), 351-355 (1985), the par locus of the plasmid RK2 described by Sobecky et al. (Journal of Bacteriology 178 (7), 2086-2093 (1996)) and Roberts and Helinsky (Journal of Bacteriology 174 (24), 8119-8132 (1992)), the par locus of the plasmid RP4 described by Eberl et al. (Molecular Microbiology 12 (1), 131-141 (1994)) and the parA locus of the plasmid R1 described by Gerdes and Molin (Journal of Molecular Biology 190 (3), 269-279 (1986)), Dam and Gerdes (Journal of Molecular Biology 236 (5), 1289-1298 (1994)) and Jensen et al. (Proceedings of the National Academy of Sciences USA 95 (15), 8550-8555 (1998).

As a result of enhancement, in particular overexpression, the activity or concentration of the corresponding protein or enzyme is generally increased by at least 10%, 25%, 50%, 75%, 100%, 150%, 200%, 300%, 400% or 500%, at most 1000% or 2000%, with respect to that of the wild type protein or to the activity or concentration of the protein in the starting microorganism.

To produce an enhancement, expression of the genes or the catalytic or functional properties of the enzymes or proteins are increased, for example. Optionally, the two measures may be combined.

Thus, for example, the copy number of the corresponding genes can be increased by at least one (1), or the promoter and regulation region or the ribosome binding site which is located upstream of the structure gene can be mutated. Expression cassettes which are incorporated upstream of the structure gene act in the same way. In addition it is possible to increase expression during the course of fermentative L-threonine production by the use of inducible promoters. Expression is also improved by measures to extend the lifetime of the m-RNA. Furthermore, the enzyme activity can also be enhanced by inhibiting degradation of the enzyme protein. The genes or gene constructs may either be present in the plasmids with different copy numbers or integrated and amplified in the chromosome. Alternatively, moreover, overexpression of the relevant genes can be achieved by modifying the composition of the medium and culture management.

The expression “attenuation” in this connection describes the reduction in or switching off of the intracellular activity or concentration of one or more enzymes or proteins in a microorganism which are coded by the corresponding DNA, for example by using a weak promoter or an open reading frame or gene or allele which codes for a corresponding enzyme with a lower activity or inactivates the corresponding enzyme or protein or gene and optionally by combining these measures.

As a result of attenuation, the activity or concentration of the corresponding protein or enzyme is generally lowered to 0 to 75%, 0 to 50%, 0 to 25%, 0 to 10%, 0 to 5% or 0 to 1% or 0 to 0.1% of the activity or concentration of the wild type protein or of the activity or concentration of the protein in the starting microorganism.

To produce an attenuation, for example, expression of the genes or open reading frames or the catalytic or functional properties of the enzymes or proteins are lowered or switched off. Optionally, the two measures may be combined.

Gene expression can be reduced by suitable culture management, by genetic modification (mutation) of the signal structures of gene expression or also by antisense-RNA techniques. Signal structures of gene expression are, for example, repressor genes, activator genes, operators, promoters, attenuators, ribosome binding sites, the start codon and terminators. Information about this can be found by a person skilled in the art, inter alia, for example in Jensen and Hammer (Biotechnology and Bioengineering 58: 191-195 (1998)), in Carrier and Keasling (Biotechnology Progress 15: 58-64 (1999)), Franch and Gerdes (Current Opinion in Microbiology 3: 159-164 (2000)) and in well-known textbooks on genetics and molecular biology, for example the textbook by Knippers (“Molekulare Genetik”, 6th edition, Georg Thieme Verlag, Stuttgart, Germany, 1995) or the book by Winnacker (“Gene und Klone”, VCH Verlagsgesellschaft, Weinheim, Germany, 1990).

Mutations which lead to a modification, for example a reduction, in the catalytic properties of enzyme proteins are disclosed in the prior art. The following may be mentioned as examples: the papers by Qiu and Goodman (Journal of Biological Chemistry 272: 8611-8617 (1997)), Yano et al. (Proceedings of the National Academy of Sciences of the United States of America 95: 5511-5515 (1998)), Wente and Schachmann (Journal of Biological Chemistry 266: 20833-20839 (1991)). Summaries and reviews may be found in well-known textbooks on genetics and molecular biology such as e.g. the book by Hagemann (“Allgemeine Genetik”, Gustav Fischer Verlag, Stuttgart, 1986).

Suitable mutations are transitions, transversions, insertions and deletions of at least one (1) base pair or nucleotide. Depending on the effect of the amino acid exchange caused by the mutation on the enzyme activity, reference is made to missense mutations or nonsense mutations. Missense mutations lead to the replacement of a given amino acid in a protein for another, wherein the amino acid replacement is in particular non-conservative. This impairs the functionality or activity of the protein and reduces it to a value of 0 to 75%, 0 to 50%, 0 to 25%, 0 to 10%, 0 to 5%, 0 to 1% or 0 to 0.1%. A nonsense mutation leads to a stop codon in the coding region of the gene and thus to the premature termination of translation. Insertions or deletions of at least one base pair in a gene lead to frame shift mutations which then means that the wrong amino acids are incorporated or translation is prematurely terminated. As a result of the mutation, a stop codon is produced in the coding region and this also leads to premature termination of translation. Deletion of at least one (1) or more codons also leads typically to the complete failure of enzyme activity or function.

Strains which are suitable for the process according to the invention are, inter alia, strain BKIIM B-3996 described in U.S. Pat. No. 5,175,107, strain KCCM-10132 described in WO 00/09660 and isoleucine-requiring mutants of the strain kat-13 described in WO 98/04715. Optionally, strains with the features mentioned, can be adapted for use in the process according to the invention, in particular by incorporating a stop codon in the rpoS gene, for example an amber codon at the site corresponding to position 33 in the amino acid sequence for the RpoS protein and simultaneously incorporating a corresponding t-RNA suppressor, for example supE.

Strains which are suitable for the process according to the invention can also be identified by determining the nucleotide sequence of the rpoS gene in a L-threonine-eliminating strain of Escherichia coli. For this purpose, the rpoS gene is cloned or amplified with the aid of the polymerase chain reaction (PCR) and the nucleotide sequence is determined. If the rpoS gene contains a stop codon then, in a second step, it is checked whether it also contains a corresponding t-RNA suppressor. Optionally, the strain with the properties described above and identified in this way is provided with one or more of the other properties specified such as overexpression of the thrA allele, attenuation of threonine degradation taking place under aerobic conditions, introduction of a mutation in the ilvA gene causing an at least partial isoleucine requirement or growth in the presence of at lest 5 g/l of threonine.

The properties and features mentioned can be transferred into the desired strain by transformation, transduction or conjugation.

In the method of transformation, isolated genetic material, typically DNA, is introduced into a target strain. In the case of bacteria of the family Enterobacteriaceae such as e.g. Escherichia coli the DNA for this purpose is incorporated in plasmid-DNA or phage-DNA and this is then transferred into the target strain. The corresponding methods and working instructions are adequately well-known from the prior art and are described in detail, for example, in the manual by J. Sambrook (Molecular Cloning, A Laboratory Manual, 2nd ed., Cold Spring Harbor Laboratory Press, Cold Spring Harbor, N.Y., 1989).

Defined mutations can be transferred into suitable strains with the aid of the method of gene or allele replacement using conditional replicating plasmids. In a defined mutation at least the position in the chromosome, preferably the exact position of the modification of the nucleobase(s) and the type of modification (replacement, i.e. transition or transversion, insertion or deletion) is known. Optionally, the corresponding DNA is first sequenced, using the normal methods. A normal method for producing a gene or allele replacement is described by Hamilton et al. (Journal of Bacteriology 171: 4617-4622 (1989)), in which the temperature-sensitive replicating pSC101 derivative pMAK705 is used. Alleles from the plasmid can be transferred to the chromosome using this method. Chromosomal alleles are transferred to the plasmid in the same way. Other methods described in the prior art, such as for example the method described by Martinez-Morales et al. (Journal of Bacteriology 181: 7143-7148 (1999)), the method described by Boyd et al. (Journal of Bacteriology 182: 842-847 (2000)) or the method described in WO 01/77345, may also be used.

This method can be used, inter alia to introduce rpoS alleles which contain for example stop codons, suppressor genes such as for example supE, attenuated tdh alleles which contain for example deletions, attenuated ilvA alleles, thrA alleles which code for “feed back” resistant aspartate kinase I—homoserine dehydrogenase I variants, the rhtA23 mutation, attenuated pck alleles, attenuated alleles of the ytfp ORFs, attenuated yjfA ORFs, attenuated poxB alleles, attenuated yjgF ORFs into the desired strains.

In the method of transduction, a genetic feature from a donor strain is transferred to a target strain using a bacteriophage. This method is part of the prior art and is described for example in textbooks such as the book by E. A. Birge (Bacterial and Bacteriophage Genetics, 4th ed., Springer Verlag, New York, USA, 2000).

In the case of Escherichia coli the bacteriophage P1 is typically used for generalized transduction (Lennox, Virology 1, 190-206 (1955). A review of methods of generalised transduction is given in the article “Generalised Transduction” by M. Masters, which is contained within the text book by F. C. Neidhard (Escherichia coli and Salmonella Cellular and Molecular Biology, 2nd ed., ASM Press, Washington, DC, USA, 1996). Practical instructions are given in the manual by J. H. Miller (A Short Course In Bacterial Genetics. A Laboratory Manual and Handbook for Escherichia coli and Related Bacteria, Cold Spring Harbor Laboratory Press, New York, USA, 1992) or the manual by P. Gerhardt “Manual of Methods for General Bacteriology” (American Society for Microbiology, Washington, D.C., USA, 1981).

Using the method of transduction, resistance-promoting or other dominant genetic properties such as for example antibiotics resistance (for example kanamycin resistance, chloramphenicol resistance, rifampicin resistance or borrelidin resistance), resistance to antimetabolites (for example α-amino-β-hydroxyvaleric acid-resistance, α-methyl-serine-resistance or diaminosuccinic acid-resistance), resistance to metabolites (for example threonine resistance, homoserine resistance, glutamic acid resistance, methionine resistance, lysine resistance or aspartic acid resistance) or also the ability to utilize saccharose can be transferred into suitable target strains.

The method of transduction is also suitable for introducing non-selectable genetic properties such as, for example, amino acid auxotrophies or requirements (for example an isoleucine requirement, methionine requirement or m-diamino pimelic acid requirement), vitamin requirements or sensitivities to antimetabolites (for example sensitivity to fluoropyruvic acid) into target strains. For this purpose, E. coli strains are used which contain the transposon Tn10 or Tn10kan on the chromosome, at spacings of approximately one minute. These strains are known under the expression “Singer Collection” or “Singer/Gross Collection” (Singer et al., Microbiological Reviews 53, 1-24, 1989). These strains are generally available from the E. coli Genetic Stock Center at Yale University (New Haven, Conn., USA). Further information can be found in the article by M. K. B. Berlyn et al. “Linkage Map of Escherichia coli K-12, Edition 9”, which is contained within the textbook by F. C. Neidhard (Escherichia coli and Salmonella Cellular and Molecular Biology, 2nd ed., ASM Press, Washington, D.C., USA, 1996). In a similar way, genetic properties which are not selectable (for example fluoropyruvic acid sensitivity, suppressor mutations) and also those where the mutation site is not known, can be transferred into a variety of strains. Instructions for this process can be found, inter alia, in the textbook by J. Scaife et al. (Genetics of Bacteria, Academic Press, London, UK, 1985), in the article mentioned above by M. Masters and in the manual mentioned above by J. H. Miller. The tetracyclin resistance gene introduced with the transposon Tn10 may optionally be removed again using the method described by Bochner et al. (Journal of Bacteriology 143, 926-933 (1980)).

In the method of conjugation, genetic material is transferred from a donor to a target by cell-cell contact. Conjugative transfer of the F-factor (F: fertility), conjugative gene transfer using Hfr strains (Hfr: high frequency of recombination) and strains which contain a F′-factor (F′:F prime), are among the classical processes of genetics. Reviews can be found, inter alia, in the standard work by F. C. Neidhard (Escherichia coli and Salmonella Cellular and Molecular Biology, 2nd ed., ASM Press, Washington, D.C., USA, 1996). Practical instructions are given for example, in the manual by J. H. Miller (A Short Course In Bacterial Genetics. A Laboratory Manual and Handbook for Escherichia coli and Related Bacteria, Cold Spring Harbor Laboratory Press, New York, USA, 1992) or the manual by P. Gerhardt “Manual of Methods for General Bacteriology” (American Society for Microbiology, Washington, DC, USA, 1981). F-, F′ and Hfr strains are generally available from the E. coli Genetic Stock Center at Yale University (New Haven, Conn., USA).

The method of conjugation was used, for example, to transfer the mutation thrC1010 described by Theze and Saint-Girons (Journal of Bacteriology 118, 990-998 (1974)) into the strain MG442 (Debabov, Advances in Biochemical Engineering/Biotechnology 79, 113-136 (2003). In the prior art, for example in Schmid et al. (Journal of Bacteriology 151, 68-76 (1982)) or Smith and Parsell (Journal of General Microbiology 87, 129-140 (1975)) and Livshits et al. (In: Conference on Metabolic Bacterial Plasmids. Tartusk University Press, Tallin, Estonia (1982), p 132-134 and 144-146,) conjugative plasmids are described which carry the ability to utilize saccharose. Thus, Debabov (In: Proceedings of the IVth International Symposium on Genetics of Industrial Microorganisms 1982. Kodansha Ltd, Tokyo, Japan, p 254-258) reports on the design of threonine-producing strains in which the ability to utilize saccharose was incorporated by using conjugation.

1-39. (canceled)

40. A process for the preparation of L-threonine using bacteria of the Enterobacteriaceae family, comprising:
a) inoculating and culturing a bacterium of the Enterobacteriaceae family in at least a first nutrient medium, said culturing taking place in a fermentation container under conditions allowing for the formation of L-threonine;
b) abstracting some of the fermentation broth from the culture prepared in step a), wherein more than 90 vol. % of the total volume of the fermentation broth remains in said fermentation container;
c) topping up the fermentation broth remaining in the fermentation container after the abstraction of step b) with at least one additional nutrient medium, wherein said additional nutrient medium contains at least one source of carbon, at least one source of nitrogen and at least one source of phosphorus, and wherein the concentration of carbon in said fermentation broth is adjusted to a maximum of 30 g/l; and
d) after the topping up of step c), continuing to culture said bacterium under conditions which allow for the formation of L-threonine.

41. The process of claim 40, wherein said culturing in step a) is carried out by a batch process.

42. The process of claim 40, wherein said culturing in step a) is performed by a fed batch process in which nutrient medium is added to said fermentation container.

43. The process of claim 40, wherein less than 2 vol. % of fermentation broth is abstracted in step b).

44. The process of claim 40, further comprising purifying said L-threonine from said fermentation broth.

45. The process of claim 40, wherein said source of carbon is one or more compounds chosen from the group consisting of: saccharose, molasses from sugar beet or sugar cane, fructose, glucose, starch hydrolysate, cellulose hydrolysate, arabinose, maltose, xylose, acetic acid, ethanol and methanol.

46. The process of claim 40, wherein said source of nitrogen comprises:
a) one or more organic nitrogen-containing substances or substance mixtures selected from the group consisting of: peptones; yeast extract; meat extract; malt extract; corn steep liquor; soy bean flour; and urea; and/or one
b) or more inorganic compounds chosen from the group consisting of: ammonia; ammonium-containing salts; and salts of nitric acid.

47. The process of claim 40, wherein said source of phosphorus is selected from the group consisting of: phosphoric acid; an alkali metal or alkaline earth metal salt or polymer of phosphoric acid; and phytic acid.

48. The process of claim 40, wherein said bacterium of the Enterobacteriaceae family is of the species Escherichia coli.

49. The process of claim 40, wherein steps b) and c) are repeated 5-30 times.

50. The process of claim 40, wherein complete topping up with nutrient media takes at most 2 hours.

51. The process of claim 40, wherein said nutrient feed medium has a phosphorus to carbon ratio (P/C ratio) selected from: not more than 4; not more than 3; not more than 2; not more than 1.5; not more than 1; not more than 0.7; not more than 0.5; not more than 0.48; not more than 0.46; not more than 0.44; not more than 0.42; not more than 0.40; not more than 0.38; not more than 0.36; not more than 0.34; not more than 0.32; and not more than 0.30.

52. The process of claim 40, wherein the culture broth removed is provided with oxygen or an oxygen-containing gas until the concentration of the source of carbon falls below a value selected from: 2 g/l; 1 g/l; and 0.5 g/l.

53. The process of claim 52, further comprising purifying said L-threonine from said fermentation broth.

54. The process of claim 53, further comprising:
a) removing at least 90% of the biomass from the culture withdrawn in step (b); and
b) then removing at least 90% of the remaining water.

55. The process of claim 40, wherein the concentration of the source of carbon during the culture is adjusted to a value selected from: not more than 20; not more than 10; not more than 5 g/l and not more than 2 g/l.

56. The process of claim 40, wherein the yield of L-threonine formed, based on the source of carbon employed, is selected from a value of: at least 31%; at least 37%; at least 42%; at least 48%.

57. The process of claim 40, wherein L-threonine is formed with a space/time yield having a value selected from: 1.5 to 2.5 g/l per h; 2.5 to 3.5 g/l per h; 3.5 to 5.0 g/l per h; and more than 8.0 g/l per h.

58. The process of claim 40, wherein said bacterium of the Enterobacteriaceae family comprises one or more of the following features:
a) a threonine-insensitive aspartate kinase I—homoserine dehydrogenase I;
b) an rpoS gene with a stop codon selected from the group consisting of: opal; ochre; and amber; and a t-RNA suppressor selected from the group consisting of: the opal suppressor; the ochre suppressor; and the amber suppressor.

59. The process of claim 58, wherein said bacterium of the Enterobacteriaceae family further comprises one or more of the following features:
a) an incapability, under aerobic culture conditions, of breaking down threonine,
b) at least a partial need for isoleucine, and
c) a capacity to grow in the presence of at least 5 g/l threonine.

60. The process of claim 58, wherein said bacterium of the Enterobacteriaceae family further comprises one or more of the following features:
a) attenuation of phosphoenol pyruvate carboxykinase, which is coded for by the pcka gene;
b) attenuation of phosphoglucose isomerase, which is coded for by the pgi gene;
c) attenuation of the YtfP gene product, which is coded for by the open reading frame ytfp;
d) attenuation of the YjfA gene product, which is coded for by the open reading frame yjfA;
e) attenuation of pyruvate oxidase, which is coded for by the poxB gene;
f) attenuation of the YjgF gene product, which is coded for by the open reading frame yjgF;
g) enhancement of transhydrogenase, which is coded for by the genes pntA and pntB;
h) enhancement of phosphoenol pyruvate synthase, which is coded for by the pps gene;
i) enhancement of phosphoenol pyruvate carboxylase, which is coded for by the ppc gene;
j) enhancement of the regulator RseB, which is coded for by the rseB gene;
k) enhancement of the galactose proton symporter, which is coded for by the galP gene;
l) an ability to use sucrose as a source of carbon;
m) enhancement of the YedA gene product, which is coded for by the open reading frame yedA;
n) growth in the presence of at least 0.1 to 0.5 mM or at least 0.5 to 1 mM borrelidin (borrelidin resistance);
o) growth in the presence of at least 2 to 2.5 g/l or at least 2.5 to 3 g/l diaminosuccinic acid (diaminosuccinic acid resistance);
p) growth in the presence of at least 30 to 40 mM or at least 40 to 50 mM α-methylserine (α-methylserine resistance);
q) growth in the presence of not more than 30 mM or not more than 40 mM or not more than 50 mM fluoropyruvic acid (fluoropyruvic acid sensitivity);
r) growth in the presence of at least 210 mM or at least 240 mM or at least 270 mM or at least 300 mM L-glutamic acid (glutamic acid resistance);
s) at least a partial need for methionine;
t) at least a partial need for m-diaminopimelic acid;
u) growth in the presence of at least 100 mg/l rifampicin (rifampicin resistance);
v) growth in the presence of at least 15 g/l L-lysine (lysine resistance);
w) growth in the presence of at least 15 g/l methionine (methionine resistance);
x) growth in the presence of at least 15 g/l L-aspartic acid (aspartic acid resistance); or
y) enhancement of pyruvate carboxylase, which is coded for by the pyc gene.
